(19) United States
(12) Patent Application Publication
(10) Pub. No.: US 2009/0008575 A1
(43) Pub. Date: Jan. 8, 2009

(54) PARTICLE IRRADIATION APPARATUS, PARTICLE BEAM IRRADIATION METHOD AND PARTICLE TREATMENT SYSTEM

(76) Inventors: Takashi OKAZAKI, Hitachinaka (JP); Hisataka Fujimaki, Nissin (JP); Shinichiro Fujitaka, Hitachi (JP); Rintaro Fujimoto, Hitachinaka (JP); Yusuke Fujii, Hitachi (JP); Kazuo Hiramoto, Hitachiohta (JP)

Correspondence Address: MATTINGLY, STANGER, MALUR & BRUNDIDGE, P.C., 1800 DIAGONAL ROAD, SUITE 370, ALEXANDRIA, VA, 22314, US

(21) Appl. No.: 12/119,787

(22) Filed: May 13, 2008

(30) Foreign Application Priority Data
May 14, 2007 (JP) 2007-127645

(51) Int. Cl. A61N 5/00

(52) U.S. Cl. 250/492.1

(57) ABSTRACT

A particle irradiation apparatus and a particle beam irradiation method that controls the energy and irradiation dose of a particle beam to form a high dose region having a high uniformity of depth-directional spread (Spread Out Bracici Peak, referred to as SOBP). A SOBP having a steep falling edge of the dose distribution on the deep side from the body surface is formed based on a method of superimposing SOBPs each having a small dose distribution width to form a desired SOBP. An energy-spread-device forms a first SOBP having a small dose distribution width; and an energy spread device 2 forms a second SOBP having a small dose distribution width and a steep falling edge of the dose distribution at the deepest portion from the body surface. The thus formed SOBPs are superimposed to form a SOBP having a length suitable for the target region.

### BACKGROUND OF THE INVENTION

1. Field of the Invention

The present invention relates to a particle irradiation apparatus, a particle beam irradiation method and a particle treatment system. More particularly, the present invention is concerned with a particle irradiation apparatus, a particle beam irradiation method and a particle treatment system which are suitable for forming a high dose region in a dose distribution by use of a ridge filter.

2. Description of the Related Art

A particle treatment system, for example, a proton-beam treatment system, is one of the effective means for cancer treatment and is expected to be more increasingly used in the future. With the proton-beam treatment, it is demanded that the dose distribution in an affected part of patient's body (target region) be controlled to a uniform or predetermined state.

Methods of controlling the dose distribution to a uniform state, etc., include using a scatterer to spread out a proton beam on a plane perpendicular to the traveling direction of the proton beam (irradiation field) or using a beam having a small proton beam radius to scan the irradiation field.

The dose distribution in the traveling direction of the proton beam (depth direction) is formed by utilizing a proton beam characteristic that the proton beam deposits the most part of its energy immediately before it stops to form a dose distribution called a Bragg curve as well as its characteristic that the depth position of a Bragg peak (a peak of the Bragg curve) can be controlled by the magnitude of the energy of the proton beam injected into the body. In that case, the energy of the proton beam is appropriately selected, and the proton beam is stopped in the vicinity of the affected part of the body, thereby applying the most part of the energy to cancer cells at the affected part of the body. Here, the depth-directional width of the Bragg peak is several millimeters. Typically, the affected part of the body has a depth-directional thickness exceeding that of the Bragg peak. In order to effectively irradiate the entire portion of such an affected part of the body in the depth direction with a particle beam, it is necessary to control the energy and irradiation dose of the proton beam so as to form a high dose region having a high uniformity of depth-directional spread and the size of the affected part of the body (Spread Out Bragg Peak, hereinafter referred to as SOBP). In order to form a dose distribution in the traveling direction (depth direction) of the proton beam, a method of using a ridge filter or an RMW (Range Modulation Wheel) or a method of changing the beam energy type from an accelerator is used to form a SOBP (refer to, for example, W. T. Chu, B. A. Ludewigt and T. R. Renner, Rev. Sci. Instrum. 64, 2055-2122, 1993).

In the formation of a SOBP, the ridge filter and RMW make it possible to form SOBPs at one time in the depth direction through irradiation with a proton beam; however, in a case where the shape of an affected part of the body changes in the depth direction, a portion other than the affected part of the body will be irradiated with the proton beam. With this method, it is necessary to prepare a number of ridge filters and RMWs according to the widths of SOBPs to be formed. Further, although the method of changing the beam energy from the accelerator makes it possible to irradiate the affected part of the body according to the shape thereof, it is necessary to prepare a number of energy types.

Further, there is another method of using a ridge filter (refer to, for example, B. Schaffner, et al. Med. Phys. 27 (4), 716-724, April 2000). With this method, an affected part of the body is sectioned into layers before irradiation, i.e., a plurality of SOBPs each having a small width according to the sectioned layers of the affected part of the body are formed and then superimposed to form a SOBP that suits the shape of the affected part of the body (hereinafter, a SOBP having a small width is referred to as a SOBP having a small dose distribution width so as to be distinguished from a SOBP having the size of the affected part of the body).

This method combines SOBPs each having a small dose distribution width to produce a SOBP having the size of the affected part of the body. Therefore, it is not necessary to prepare a ridge filter for each SOBP length, thereby reducing the number of ridge filters. In order to form SOBPs each having a small dose distribution width for performing this irradiation, a plurality of energy spread devices having a spread-out Bragg peak width and a controlled peak intensity are applied (Ridge filters and other apparatuses used to spread out the energy in the depth direction are collectively referred to as energy spread devices).

### SUMMARY OF THE INVENTION

However, with the method of forming SOBPs each having a small dose distribution width and combining them to form a desired SOBP, if the Bragg peak width is spread out in order to reduce the number of layers and accordingly shorten irradiation time, the dose distribution at a boundary of a SOBP on the deep side of the irradiation region is not sharp, and there arises a problem that the falling edge of the dose distribution on the deep side from the body surface becomes not steep. If the falling edge of the dose distribution on the deep side from the body surface is not steep, normal tissues other than the affected part of the body will be irradiated. If SOBPs each having a small dose distribution width are produced in order to avoid this, there also arises a problem that the number of layers increases, resulting in a prolonged irradiation time.

An object of the present invention is to provide a particle irradiation apparatus, a particle beam irradiation method and a particle treatment system which make it possible to form a combined SOBP having a steep falling edge of the dose distribution on the deep side from the body surface and perform beam irradiation in a short time based on a method of superimposing SOBPs each having a small dose distribution width to form a desired SOBP.

In order to accomplish the above-mentioned object, the present invention provides a particle irradiation apparatus which irradiates a target region with a particle beam, wherein a plurality of energy spread devices for forming SOBPs having different dose distributions are used to combine depth-directional dose distributions.

In order to accomplish the above-mentioned object, the present invention provides a particle irradiation apparatus which irradiates a target region with a particle beam, wherein a plurality of energy spread devices having different geometric shapes are used to combine depth-directional dose distributions.

Preferably, the particle irradiation apparatus comprises: a monitor which measures an irradiation dose; and a control unit which controls the irradiation dose; wherein the particle irradiation apparatus performs: sectioning a target region into layers; determining an energy spread device and an irradiation dose used for each layer; measuring an irradiation dose for each layer by means of the monitor; and irradiating respective regions with the irradiation dose controlled by the control unit and combining the produced SOBPs each having a small dose distribution width.

Preferably, the particle irradiation apparatus further comprises: an energy spread device which forms a first SOBP having a small dose distribution width; and an energy spread device which forms a second SOBP having a small dose distribution width at the deepest portion of the target region from the body surface; wherein the particle irradiation apparatus performs: irradiating the target region with the particle beam through the use of the energy spread device to form the second SOBP having a small dose distribution width at the deepest portion of the target region from the body surface; and irradiating with the particle beam the range from the second deepest portion to the shallowest target region on the body surface side through the use of the energy spread device once or a plurality of times to form first SOBPs each having a small dose distribution width whereby the formed SOBPs are superimposed to form a SOBP having a length suitable for the target region.

Here, one type of an energy spread device corresponds to an energy spread device for producing a SOBP having a dose distribution width smaller than that produced by an ordinary energy spread device, and the other type of energy spread device corresponds to an energy spread device for forming a SOBP having a steep falling edge of the dose distribution on the deep side from the body surface.

Preferably, the particle irradiation apparatus further comprises: a range shifter which changes the irradiation depth of the particle beam; a range-shifter drive unit which drives the range shifter; and one or a plurality of energy types as particle beam energy of an accelerator; wherein the particle irradiation apparatus performs: forming high dose regions by changing the irradiation depth of the particle beam by use of either the range shifter driven by the range-shifter drive unit or the energy type or both, through the use of the above-mentioned one type of energy spread device and the other type of energy spread device whereby the formed high dose regions are superimposed.

Further, in order to accomplish the object, the present invention provides a particle beam irradiation method of irradiating a target region with a particle beam, wherein the method comprises the steps of: irradiating the target region with the particle beam through the use of an energy spread device to form a second SOBP having a small dose distribution width at the deepest portion of the target region from the body surface; and irradiating with the particle beam the range from the second deepest portion to the shallowest target region on the body surface side through the use of an energy spread device 1 once or a plurality of times to form first SOBPs each having a small dose distribution width whereby the formed SOBPs are superimposed to form a SOBP having a length suitable for the target region.

This method makes it possible to form a SOBP having a steep falling edge of the dose distribution on the deep side from the body surface based on the method of superimposing SOBPs each having a small dose distribution width to form a desired SOBP.

Further, in order to accomplish the object, the present invention provides a particle beam irradiation method of irradiating a target region with a particle beam, wherein the method comprises the steps of: directly irradiating the target region with the particle beam without using an energy spread device to form a SOBP having a small dose distribution width at the deep portion of the target region from the body surface; and irradiating with the particle beam the range from the second deepest portion to the shallowest target region on the body surface side through the use of the energy spread device 1 once or a plurality of times to form SOBPs each having a small dose distribution width whereby the formed SOBPs are superimposed to form a SOBP having a length suitable for the target region.

This method makes it possible to form a SOBP having a steep falling edge of the dose distribution on the deep side from the body surface based on the method of superimposing SOBPs each having a small dose distribution width to form a desired SOBP.

Further, in order to accomplish the object, the present invention provides a particle treatment system, comprising: an accelerator which accelerates a particle beam; and a particle irradiation apparatus which receives the particle beam from the accelerator and irradiates a target region with the particle beam; wherein the particle irradiation apparatus comprises: a first energy spread device; and a second energy spread device which forms a steeper dose distribution in the traveling direction of the particle beam than that formed by the first energy spread device; wherein at least the first and second energy spread devices are employed to combine dose distributions in the traveling direction of the particle beam.

Preferably, the particle treatment system further comprises: a control unit which performs control so as to arrange the second energy spread device on a path of the particle beam when a layer at the deepest portion, out of a plurality of sectioned layers of the target region, is irradiated with the particle beam. Preferably, the particle beam is a proton beam. The particle beam may be a heavy particle beam.

In accordance with the present invention, it becomes possible to form a SOBP having a steep falling edge of the dose distribution on the deep side from the body surface based on the method of superimposing SOBPs each having a small dose distribution width to form a desired SOBP.

### BRIEF DESCRIPTION OF THE DRAWINGS

FIG. 1 is a block diagram showing the configuration of a particle irradiation apparatus according to a first embodiment of the present invention.

FIG. 2A is a diagram showing the configuration of an ordinary energy spread device used for an energy-spread-device section of the particle irradiation apparatus according to the first embodiment of the present invention;

FIG. 2B, a diagram showing a function of the ordinary energy spread device.

FIG. 3A is a diagram showing the configuration of an energy spread device for producing a SOBP having a small dose distribution width used for the energy-spread-device section of the particle irradiation apparatus according to the first embodiment of the present invention;

FIGS. 3B and 3C, diagrams showing functions of the energy spread device.

FIG. 4 is a perspective view showing the configuration of the energy spread devices for producing a SOBP having a small dose distribution width used for the energy-spread-device section of the particle irradiation apparatus according to the first embodiment of the present invention.

FIGS. 5A and 5B are diagrams showing functions of energy spread devices for forming a SOBP having a steep falling edge of the dose distribution on the deep side from the body surface used for the energy-spread-device section of the particle irradiation apparatus according to the first embodiment of the present invention.

FIG. 6 is a plan view showing a first configuration of the energy-spread-device section of the particle irradiation apparatus according to the first embodiment of the present invention.

FIG. 7 is a plan view showing a second configuration of the energy-spread-device section of the particle irradiation apparatus according to the first embodiment of the present invention.

FIG. 8 is a diagram explaining a process of SOBP formation by use of the particle irradiation apparatus according to the first embodiment of the present invention.

FIGS. 9A and 9B are diagrams explaining SOBPs formed by the particle irradiation apparatus according to the first embodiment of the present invention.

FIGS. 10A and 10B are diagrams explaining SOBPs produced by the energy spread device for producing a SOBP having a small dose distribution width used for a particle irradiation apparatus according to a second embodiment of the present invention.

FIGS. 11A and 11B are diagrams explaining SOBPs produced by the energy spread device for producing a SOBP having a small dose distribution width used for a particle irradiation apparatus according to a third embodiment of the present invention.

### DESCRIPTION OF NUMERALS

1 . . . Proton beam
2 . . . Monitor
3 . . . Scanning magnet
4 . . . Scatterer
5 . . . Energy-spread-device section
6 . . . Range shifter
7 . . . Dose monitor
8 . . . Block collimator
9 . . . Patient collimator
10 . . . Isocenter
20 . . . Control unit
22 . . . Energy-spread-device drive unit
24 . . . Range-shifter drive unit
40 . . . Rack
42 . . . Holder
RF . . . Ordinary energy spread device
M-RF . . . Energy spread device for producing a SOBP having a small dose distribution width
SM-RF . . . Energy spread device for forming a SOBP having a steep falling edge of the dose distribution on the deep side from the body surface.

### DETAILED DESCRIPTION OF THE PREFERRED EMBODIMENTS

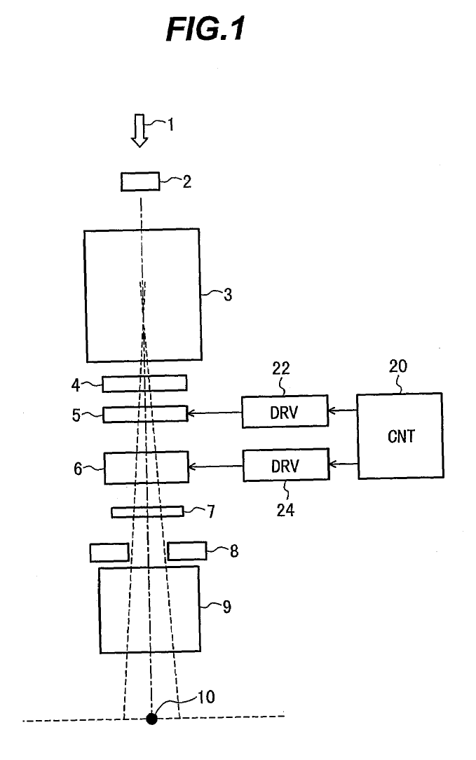

The configuration and operations of a particle irradiation apparatus according to a first embodiment of the present invention will be explained below with reference to FIGS. 1 to 9.

First of all, the configuration of the particle irradiation apparatus according to the present embodiment will be explained below with reference to FIG. 1. Here, a particle irradiation apparatus of a proton-beam treatment system will be explained as an example. FIG. 1 shows the general configuration of an irradiation nozzle portion of the proton-beam irradiation apparatus in the proton-beam treatment system.

FIG. 1 is a block diagram showing the configuration of the particle irradiation apparatus according to the first embodiment of the present invention.

A proton beam 1 enters the proton-beam irradiation apparatus (irradiation nozzle) from the accelerator side. The proton-beam irradiation apparatus comprises: a monitors 2 such as a beam profile monitor, a scanning magnet 3 for the proton beam; a scatterer 4 for spreading the diameter of the proton beam; an energy-spread-device section 5; a range shifter 6; a dose monitor 7; a block collimator 8; a patient collimator 9; a control unit 20; an energy-spread-device drive unit 22; and a range-shifter drive unit 24. An isocenter 10 denotes the point in space through which the central ray of the proton beam passes to irradiate an affected part of patient's body.

The scatterer 4 is used to widen the beam diameter of the proton beam 1. The smaller the beam radius, the higher becomes beam utilization efficiency, but at the same time the higher becomes beam positional accuracy. In contrast, the larger the beam radius, the lower becomes the beam utilization efficiency, but at the same time the lower becomes the beam positional accuracy. Here, a beam radius widened by the scatterer 4 is larger than the diameter of a pencil beam.

Methods of irradiating an irradiation field perpendicular to the traveling direction of the proton beam include the use of the scanning magnet 3 to perform spot scanning, raster scanning, or multi wobbler scanning by the proton beam or the use of double scattering method.

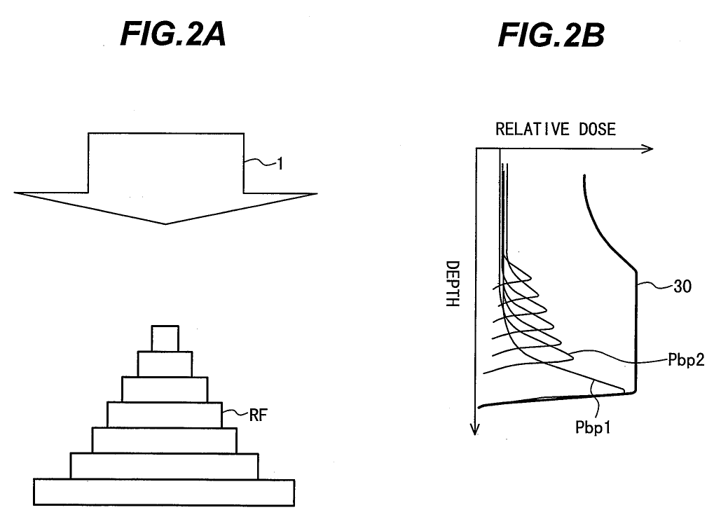

In the energy-spread-device section 5, various energy spread devices are replaceably arranged for the proton beam. Although mentioned later with reference to FIGS. 2 to 5, the various energy spread devices include an ordinary energy spread device, an energy spread device for producing a SOBP having a smaller dose distribution width than that produced by the ordinary energy spread device, an energy spread device for forming a SOBP having a steep falling edge of the dose distribution on a deeper side from the body surface, etc. The energy-spread-device drive unit 22 changes the types of the energy spread devices to be inserted into the proton beam based on a control command from the control unit 20.

The range shifter 6 is used to shift the position (from the body surface) of a SOBP having a small dose distribution width produced by the energy spread device for producing such a SOBP and by the energy spread device for forming a SOBP having a steep falling edge of the dose distribution on a deeper side from the body surface. The range shifter 6 is composed of, for example, range shifter plates having different thicknesses. The thickness of the range shifter plates is based on a binary system in which the thickness increases, multiplied by 2, for example, 1 mm, 2 mm, 4 mm, 8 mm, 16 mm, 32 mm, and so on. For example, the use of two range shifter plates having a thickness of 2 mm and 8 mm, respectively, results in the range shifter 6 having a thickness of 10 mm, and the use of two range shifter plates having a thickness of 4 mm and 16 mm, respectively, results in the range shifter 6 having a thickness of 20 mm. The range-shifter drive unit 24 selects range shifter plates to be inserted into the proton beam based on a control command from the control unit 20 and then inserts the selected range shifter plates into the proton beam.

The configurations of the various energy spread devices used for the energy-spread-device section 5 of the particle irradiation apparatus according to the present embodiment will be explained below with reference to FIGS. 2 to 5.

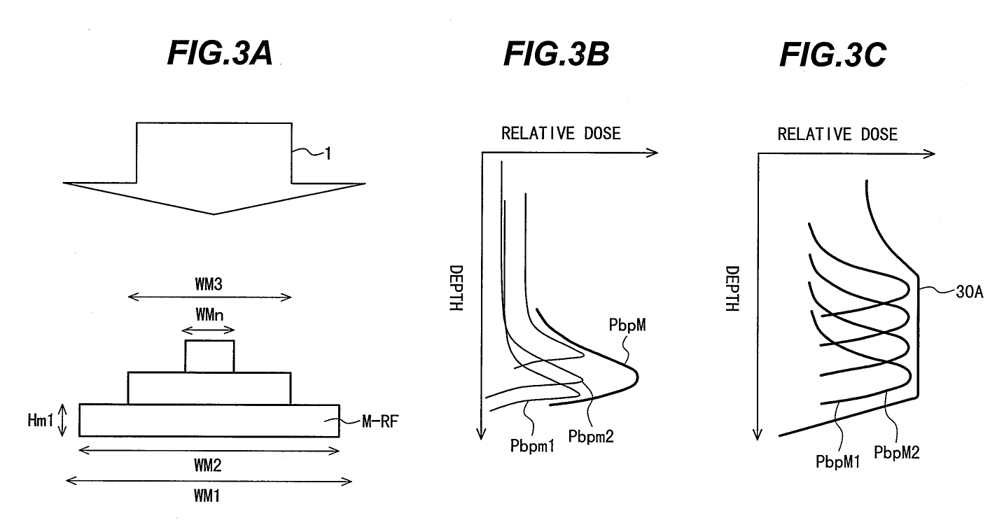

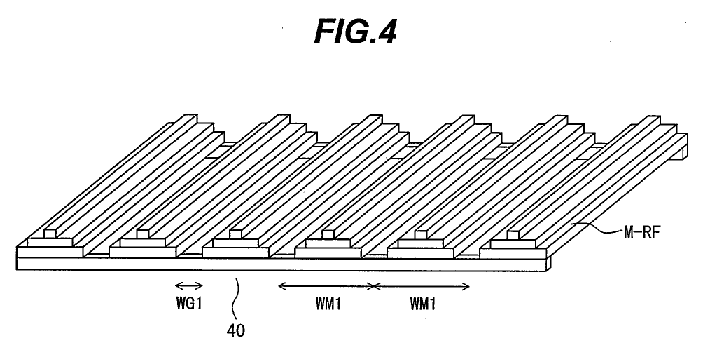

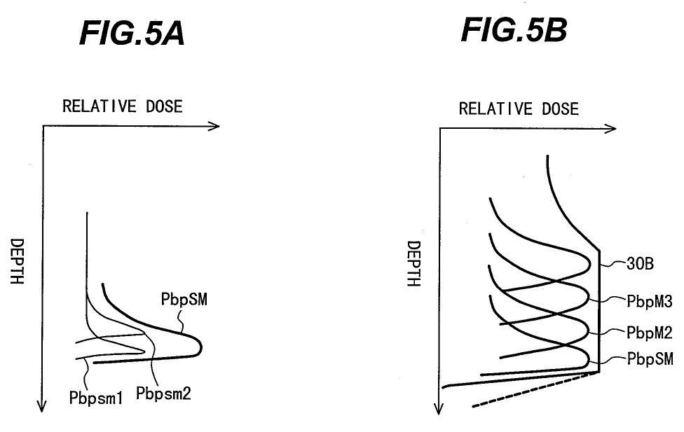

FIG. 2A is a diagram showing the configuration of the ordinary energy spread device used for the energy-spread-device section of the particle irradiation apparatus according to the first embodiment of the present invention; FIG. 2B, a diagram showing a function of the ordinary energy spread device. FIG. 3A is a diagram showing the configuration of the energy spread device for producing a SOBP having a small dose distribution width used for the energy-spread-device section of the particle irradiation apparatus according to the first embodiment of the present invention; FIGS. 3B and 3C, diagrams showing functions of the energy spread device. FIG. 4 is a perspective view showing the configuration of the energy spread devices for producing a SOBP having a small dose distribution width used for the energy-spread-device section of the particle irradiation apparatus according to the first embodiment of the present invention. FIGS. 5A and 5B are diagrams showing functions of the energy spread device for forming a SOBP having a steep falling edge of the dose distribution on a deeper side from the body surface used for the energy-spread-device section 5 of the particle irradiation apparatus according to the first embodiment of the present invention.

First of all, the ordinary energy spread device will be explained with reference to FIGS. 2A and 2B. As shown in FIG. 2A, an ordinary energy spread device RF for forming a SOBP is configured such that an upper layer has a smaller width than the layer right beneath it. Although FIG. 2A shows 7 layers as an example, the ordinary energy spread device, in fact, includes about 30 layers. The example shown is a schematic diagram, and the number and width of layers differ from those of an actual energy spread device.

As shown in FIG. 2A, the energy spread device RF is irradiated with the proton beam 1, and the arrival positions of the proton beam differ for each layer of the energy spread device RF having a different height. As a result, as shown in FIG. 2B, Bragg peak positions Pbp1, Pbp2, and so on are formed at different depths from the body surface. The fewer number of layers the proton beam passes through, the deeper portion from the body surface reaches a Bragg peak position Pbp. The Bragg peak positions Pbp1, Pbp2, and so on are superimposed to form a SOBP 30.

Next, the energy spread device for producing a SOBP having a small dose distribution width will be explained below with reference to FIG. 3. An energy spread device M-RF which produces a SOBP having a small dose distribution width shown in FIG. 3A has a fewer number of layers and a lower height than the ordinary energy spread device RF shown in FIG. 2. Although FIG. 3A shows 3 layers as an example, this example is a schematic diagram, and the number and width of layers differ from those of an actual energy spread device. The energy spread device M-RF is formed such that the width thereof including spaces on both sides thereof is WM1, the widest, the width of a first layer is WM2, the second widest, and the width of a second layer is WM3, the third widest. A height Hm1 is determined according to the shape of a SOBP having a small dose distribution width.

As shown in FIG. 3A, the energy spread device M-RF is irradiated with the proton beam 1, and the arrival positions of the proton beam differ for each layer of the energy spread device M-RF having a different height. As a result, as shown in FIG. 3B, Bragg peak positions Pbpm1, Pbpm2, and so on are formed at different depths from the body surface. The fewer layers the proton beam passes through, the deeper portion from the body surface reaches a Bragg peak position Pbpm. The Bragg peak positions Pbpm1, PBpm2, and so on are superimposed to form a SOBP (PbpM) having a small dose distribution width.

Then, as shown in FIG. 3C, the energy spread device M-RF is employed to produce a first SOBP (PbpM1) having a small dose distribution width. Next, the range shifter 6 explained in FIG. 1 is used, or the energy type of an accelerator is changed, or both are employed to produce a SOBP (PbpM2) having a small dose distribution width by shifting its SOBP position from the body surface. Thus, SOBPs each having a small dose distribution width are superimposed to form a SOBP 30A. The energy spread device for producing a SOBP having a small dose distribution width produces SOBPs each having a small dose distribution width and superimposes them so as to obtain a desired shape of the SOBP 30A. Accordingly, the number, width, and height of layers of the energy spread device need be adjusted so as to obtain the target shape of the SOBP. The shapes of SOBPs having small dose distribution widths are determined so as to ensure a desired SOBP flatness by superimposing the SOBPs each having a small dose distribution width.

As shown in FIG. 4, the energy spread devices for producing SOBPs each having a small dose distribution width to be used in the energy-spread-device section 5 have a configuration in which a plurality of the energy spread devices M-RF shown in FIG. 3 are arranged and fixed at equal intervals on a rack 40. If the width of each energy spread device M-RF is defined as WM1, the space width WG1 shown in FIG. 4 equals a sum total of the spaces formed on both sides of an energy spread device. It is also possible to arrange energy spread devices on a circular rack to make the whole shape circular.

These SOBPs (PbpM) each having a small dose distribution width are superimposed with each Bragg peak spread out; therefore, the falling edge of the dose distribution on a deeper side from the body surface shown in FIG. 3C is not steep. In order to avoid this, the energy spread device for forming a SOBP having a steep falling edge of the dose distribution on a deeper side from the body surface (to be explained in FIG. 5) is employed.

An energy spread device SM-RF for forming a SOBP having a steep falling edge of the dose distribution on a deeper side from the body surface will be explained below with reference to FIG. 5. The energy spread device SM-RF has larger spaces on both sides thereof than those of the energy spread device M-RF of FIG. 3A. With each layer of the energy spread device SM-RF, like FIG. 3A, the width is gradually decreased from the first layer, but at a different width ratio of each layer from that of the energy spread device M-RF.

With the energy spread device M-RF, the proton beam that reaches a deeper side of a target region from the body surface passes through the narrower spaces of the energy spread device M-RF of FIG. 3A; however, with the energy spread device SM-RF, the spaces are widened to increase the intensity of the beam that passes therethrough, thus increasing the intensity of a Bragg curve that reaches a deeper side of the target region from the body surface. The intensities of the proton beams that pass through each layer of the energy spread device SM-RF changes according to the width ratio of each layer, and the more layers a Bragg curve passes through, the shallower side of the target region from the body surface reaches a Bragg peak position. As a result, as shown in FIG. 5A, Bragg curves Pbpsm1, Pbpsm2, and so on are superimposed to produce a SOBP (PbpSM) having a steep falling edge of the dose distribution on the deep side from the body surface and having a moderate falling edge of the dose distribution on the shallow side therefrom.

With this SOBP (PbpSM), the dose distribution on the deep side of the target region from the body surface has a steep falling edge, and the dose distribution on the shallow side from the body surface has a shape that ensures the flatness of a SOBP to be formed on an affected part of the body when the SOBP (PbpSM) is combined with SOBPs (PbpM) each having a small dose distribution width produced by the energy spread device M-RF.

FIG. 5B shows SOBPs formed on an affected part of the body. On a deeper side of the target region from the body surface, a SOBP (PbpSM) having a steep falling edge of the dose distribution is formed using the energy spread device SM-RF. At shallower portions therefrom, a SOBP (PbpM2) having a small dose distribution width is produced using the energy spread device M-RF explained in FIGS. 3 and 4. Further, the range shifter 6 is used, or the energy type of the accelerator is changed, or both are employed to repetitively produce SOBPs (PbpM2, PbpM3, . . . ) each having a small dose distribution width, thus forming a SOBP 30B. In this case, the SOBP 30B having a desired length is produced by controlling the number of production repetitions of SOBPs (PbpM2, PbpM3, and so on) each having a small dose distribution width. The width of a SOBP produced by the energy spread device for forming a SOBP having a steep falling edge of the dose distribution on the deep side from the body surface is determined in relation to the width of a SOBP produced by the energy spread device M-RF used for the combination with the former SOBP so as to be preferable for producing a SOBP having an arbitrary length.

Similarly to the case of the energy spread device M-RF shown in FIG. 4, the energy spread devices for producing SOBPs each having a steep falling edge of the dose distribution on a deeper side from the body surface to be used in the energy-spread-device section 5 have a configuration in which a plurality of the energy spread devices SM-RF each having the function shown in FIG. 5 are arranged and fixed at equal intervals on the rack 40.

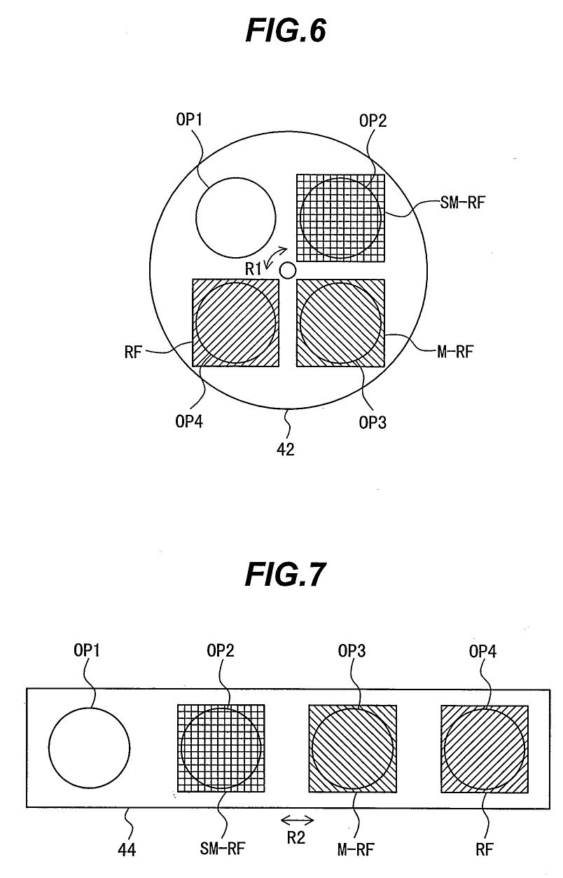

The configuration of the energy-spread-device section 5 of the particle irradiation apparatus according to the present embodiment will be explained below with reference to FIGS. 6 and 7.

FIG. 6 is a plan view showing a first configuration of the energy-spread-device section of the particle irradiation apparatus according to the first embodiment of the present invention. FIG. 7 is a plan view showing a second configuration of the energy-spread-device section of the particle irradiation apparatus according to the first embodiment of the present invention.

Referring to FIG. 6, a rotary energy spread device holder 42 has four circular openings OP1, OP2, OP3, and OP4 formed thereon. The energy spread device holder 42 can rotate in the directions shown by an arrow R1 by the energy-spread-device drive unit 22 of FIG. 1. A rack with a plurality of the energy spread devices SM-RF explained in FIG. 5 arranged thereon is placed in the circular opening OP2. The energy spread devices M-RF explained in FIG. 4 are placed in the circular opening OP3. A rack with a plurality of the energy spread devices RF explained in FIG. 2 arranged thereon is placed in the circular opening OP4. No energy spread device is placed in the circular opening OP1; the opening OP1 is used to directly irradiate the irradiation field with the proton beam. Although the four openings are placed in the example of FIG. 6, the number of openings will be increased to arrange different types of energy spread devices SM-RF and energy spread devices M-RF.

In order to produce a SOBP, for example, the holder 42 is rotated so as to position the circular opening OP2 at the position of the proton beam, and the proton beam is applied using the energy spread device SM-RF. Then, the energy spread device holder 42 is rotated, and the proton beam is applied using the energy spread device M-RF. Further, in this state, the range shifter 6 is used, or the energy type of the accelerator is changed, or both are employed to repeat irradiation while changing its depth-directional irradiation position, thus producing SOBPs.

Further, when the ordinary energy spread device RF is employed, it is possible to rotate the energy spread device holder 42 so as to set it at the position of the energy spread device RF, and irradiate the energy spread device RF with a proton beam. After SOBPs are formed using the ordinary energy spread device RF, it is possible to replace it with the energy spread device M-RF and then add SOBPs each having a small dose distribution width to form a SOBP having a large width.

Further, it is possible to directly irradiate the irradiation field with the proton beam by rotating the holder 42 so as to position the circular opening OP1 at the position through which the proton beam passes. A desired SOBP can be formed by combining SOBPs generated by the energy spread devices M-RF, the energy spread devices SM-RF, etc.

The above-mentioned configuration makes it easier to change various energy spread devices, and the use of the circular holder makes it possible to reduce a space necessary to change energy spread devices.

FIG. 7 shows the second holder configuration. An energy spread device holder 44 which is a rectangle can be slid in the directions shown by an arrow R2 by the energy-spread-device drive unit 22 shown in FIG. 1. The energy spread device holder 44 has four circular openings OP1, OP2, OP3, and OP4 formed thereon. A rack with a plurality of the energy spread devices SM-RF explained in FIG. 5 arranged thereon is placed in the circular opening OP2. The energy spread devices M-RF explained in FIG. 4 are placed in the circular opening OP3. A rack with a plurality of the energy spread devices RF explained in FIG. 2 arranged thereon is placed in the circular opening OP4. No energy spread device is placed in the circular opening OP1; the opening OP1 is used to directly irradiate the irradiation field with the proton beam.

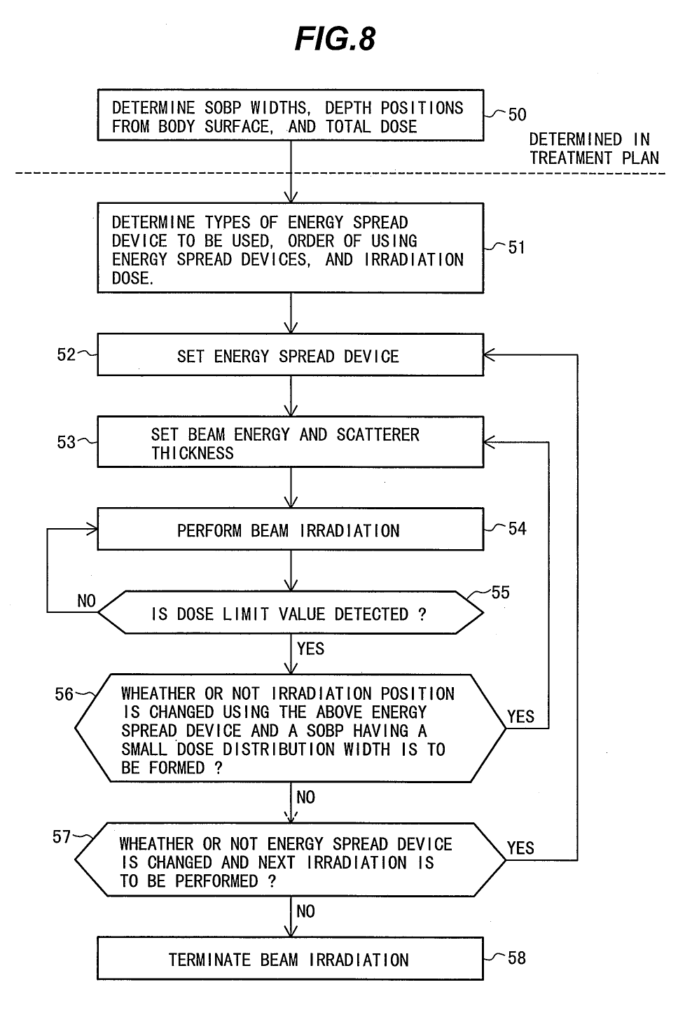

Operations of each component for SOBP formation by use of the particle irradiation apparatus according to the present embodiment will be explained below with reference to FIG. 8.

FIG. 8 is a diagram explaining operations of each component for SOBP formation by the particle irradiation apparatus according to the first embodiment of the present invention.

First of all, Step 50 determines a SOBP having the size of a target region (size of an affected part of the body), depth positions from the body surface, total irradiation dose, etc. These are determined according to a treatment plan, etc. Based on the determined values, Step 51 determines the types of energy spread devices to be used, the order of using the energy spread devices when a SOBP having the size of the target region is split into SOBPs each having a small dose distribution width, and an irradiation dose to be applied for each SOBP having a small dose distribution width.

Then, Step 52 sets an energy spread device. If this energy spread device is, for example, the energy spread device SM-RF, Step 52 sets the device. Step 53 sets beam energy according to the position from the body surface that the beam reaches and also sets the thickness of the scatterer in relation to the beam size. Step 54 is to irradiate a beam based on this condition.

Step 55 measures an irradiation dose by use of a monitor such as a dose monitor and determines whether or not the measured dose has reached a predetermined irradiation dose. If not, the beam continues to be applied until the dose limit value is detected to form a SOBP having a small dose distribution width. The control unit determines whether or not the measured dose has reached the predetermined irradiation dose.

If the dose limit value is detected, Step 56 determines whether or not the irradiation position is changed and then a SOBP having a small dose distribution width is to be formed using the above energy spread device. If this energy spread device is, for example, the energy spread device SM-RF and if it is predetermined that one SOBP having a small dose distribution width preferably be formed at the deepest portion of the affected part from the body surface by using that energy spread device, Step 56 determines that it is not necessary to change the irradiation position and form a SOBP having a small dose distribution width using the same energy spread device.

Then, the processing proceeds to Step 57 which determines whether or not the energy spread device is changed and the next irradiation is to be performed. If Step 57 determines that the energy spread device is changed according to the predetermined order of using energy spread devices and the next irradiation is to be performed, Step 52 sets an energy spread device. If this energy spread device is, for example, the energy spread device M-RF for producing a certain dose distribution width, Step 52 sets the device.

Subsequently, the same operations as above are performed (Step 53 and 54). Then, if the dose limit value is detected, Step 56 determines whether or not the irradiation position is changed and then a SOBP having a small dose distribution width is to be formed using the same energy spread device. If this energy spread device is, for example, the energy spread device M-RF and if SOBPs each having a small dose distribution width produced by the energy spread device M-RF are to be superimposed to form a desired SOBP, Step 56 determines that it is necessary to change the irradiation position and form a SOBP having a small dose distribution width using the same energy spread device. Accordingly, Step 53 sets beam energy and scatterer thickness so as to produce a SOBP having a small dose distribution width at the next position. Subsequently, Steps 54 and 55 repeat the same operations as above and followed by Step 56.

If Step 56 determines that it is not necessary to change the irradiation position and form a SOBP having a small dose distribution width using the same energy spread device, the processing proceeds to Step 57 which determines whether or not the energy spread device is changed according to the predetermined order of using the energy spread devices and the next irradiation is to be performed. If this energy spread device is, for example, the energy spread device M-RF for producing another dose distribution width, Step 52 sets the device. Subsequently, the same operations as above are performed. If Step 57 determines that it is not necessary to perform the next irradiation according to the predetermined order of using the energy spread devices, Step 58 terminates the beam irradiation. This completes the formation of the SOBP having the size of the target region by the particle irradiation apparatus.

A method of setting the beam energy will be explained below. The depth position of a SOBP is determined by the beam energy. Methods of changing the beam energy include changing the energy type by use of the accelerator or the range shifter or both. The beam energy is set by use of either of or both of those means. For example, if the position is to be changed only with the energy type of the accelerator, the number of energy types will tremendously increase and therefore the range shifter is also used. Further, when a SOBP is formed, if the position of a SOBP having a small dose distribution width is moved only with the range shifter without changing the energy type from the accelerator, the range adjustment width of the range shifter increases, thus increasing the size of the range-shifter drive unit.

Therefore, the present embodiment uses an energy type from a plurality of accelerators and the range shifter together when a SOBP is to be formed. When a SOBP having a small dose distribution width is to be formed at the deepest portion of the target region through the use of the energy spread device SM-RF for forming a SOBP having a steep falling edge of the dose distribution on the deep side from the body surface, an energy type which reaches the deepest portion of the target region is selected out of the prepared energy types from the accelerators, and irradiation is performed. If there is no suitable energy type, beam energy that reaches the deepest portion of the target region is formed using an energy type from the accelerators and the range shifter, and irradiation is performed.

When a SOBP having a small dose distribution width is to be formed at a shallower position from the deepest portion of the target region through the use of the energy spread device M-RF, similarly to the above case, beam energy that reaches the shallower position from the deepest portion of the target region is formed using the combination of the energy type from the accelerators and the range shifter, and irradiation is performed. With a similar method, SOBPs each having a small dose distribution width are repetitively produced by moving the position (in the target region) of a SOBP each having a small dose distribution width. Then, these SOBPs each having a small dose distribution width are superimposed to form a SOBP having a length suitable for the target region.

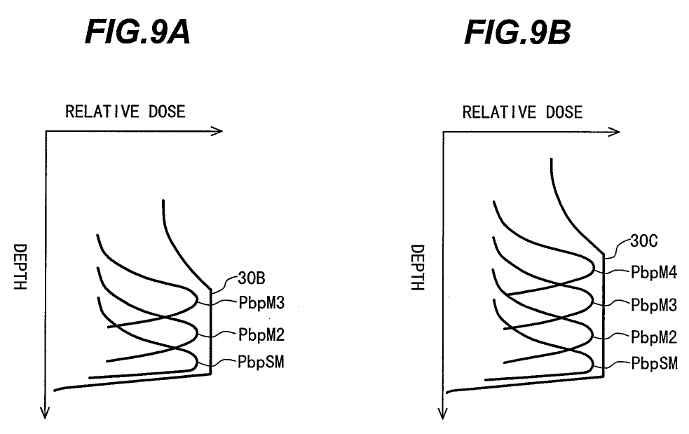

FIG. 9 explains the formation of a SOBP by use of the particle irradiation apparatus according to the present embodiment. In this case, a SOBP having a steep falling edge of the dose distribution on the deep side from the body surface and a SOBP having a small dose distribution width are used for the explanation.

FIGS. 9A and 9B are diagrams explaining SOBPs formed by the particle irradiation apparatus according to the first embodiment of the present invention. In this case, a SOBP having a steep falling edge of the dose distribution on the deep side from the body surface and a SOBP having a small dose distribution width are used.

Each component is operated in accordance with the process shown in FIG. 8 to form on the deep side of the target region from the body surface a SOBP (PbpSM) having a small dose distribution width through the use of an energy spread device for forming a SOBP having a steep falling edge of the dose distribution on the deep side from the body surface, as shown in FIG. 9A. At shallower portions therefrom, SOBPs (PbpM2, PbpM3, and so on) each having a small dose distribution width are repetitively produced using a combination of the energy type from the accelerators and the range shifter and also using an energy spread device for producing a SOBP having a small dose distribution width, and then the number of the repetitions is controlled to form a SOBP 30B. When SOBPs each having a small dose distribution width are repetitively produced, plural, different types of energy spread devices for producing a SOBP having a small dose distribution width may be used instead of using the same energy spread device.

The degree of the steepness of the falling edge of the dose distribution of the SOBP 30B on the deep side of the target region from the body surface may be represented by, for example, the distance between an 80% dose and a 20% dose (distal fall-off) on the deep (distal) side assuming that a flat portion of the dose distribution has a 100% dose. With the use of an energy spread device for forming a SOBP having a steep falling edge of the dose distribution on the deep side from the body surface, it is possible to change the moderate falling edge formed by the energy spread device for producing a SOBP having a small dose distribution width, as shown by the dashed line of FIG. 5B, to a steep falling edge, as shown by the solid line thereof. As shown in FIG. 9A, with the use of SOBPs formed by the energy spread device for forming a SOBP having a steep falling edge of the dose distribution on the deep side from the body surface, at the position of the distal fall-off, it is possible to obtain the almost same effect as that obtained when the conventional energy spread devices RF are used.

In accordance with the present embodiment, it is possible to form a desired SOBP by repeating a SOBP having a small dose distribution width almost the same times as or a fewer times than when the conventional energy spread devices M-RF are used, without degrading the steepness of the falling edge of the SOBP dose distribution on the deep side of the target region from the body surface, thus obtaining an effect that the irradiation time can be shortened. Further, since it is possible to reduce the number of layers and increase the thickness of each layer, it is robust in depth-directional movement of each layer portion and therefore suitable for irradiation in synchronization with respiration.

When the conventional energy spread devices RF are used, it is necessary to prepare the devices according to the lengths of SOBPs, thus leading to an increase in the number of the devices. In accordance with the present invention, in contrast, the SOBP length can be changed by repetitively using the energy spread device for producing a SOBP having a small dose distribution width. Therefore, it is possible to produce a desired SOBP through the use of two different types of devices: an energy spread device for producing a SOBP having a small dose distribution width and an energy spread device for forming a SOBP having a steep falling edge of the dose distribution on the deep side from the body surface. Further, even if a plurality of different types of energy spread devices for producing a SOBP having a small dose distribution width and a plurality of different types of energy spread devices for forming a SOBP having a steep falling edge of the dose distribution on the deep side from the body surface shown below are prepared, the total number of devices to be prepared will be remarkably reduced in comparison with a case of the conventional energy spread devices RF. In this way, the present invention provides an effect of remarkably reducing the number of energy spread devices in comparison with a conventional case.

Further, with the conventional energy spread devices RF, it was necessary to prepare a plurality of types of energy spread devices and change the energy spread devices RF according to the SOBP length.

The present invention makes it possible to reduce the number of changes of energy spread devices in comparison with a conventional case, and the energy spread device holder 42 makes it easier to change various energy spread devices with the above-mentioned configuration, thus providing an effect that an apparatus for changing energy spread devices for forming a SOBP can be simplified.

As mentioned above, if a plurality of energy types from accelerators are prepared to change the energy type during SOBP production, it is possible to narrow the range adjustment width of the range shifter and accordingly reduce the number of range shifter plates, thus providing an effect that the range-shifter drive unit can be simplified.

Further, in response to a change of the shape of an energy spread device, etc., it is not even necessary to modify a huge number of conventional energy spread devices RF. In this case, it is only necessary to modify several energy spread devices for producing a SOBP having a small dose distribution width, which thus provides an effect that flexible measures can be taken.

Further, as shown in FIG. 9B, it is possible to form a SOBP 30C with its length controlled by controlling the number of repetitions of SOBPs (PbpM2, PbpM3, PbpM4, and so on) each having a small dose distribution width formed by the energy spread device for producing a SOBP having a small dose distribution width. The SOBP length depends on the size of an affected part of the body and is so far changed in 1-cm steps in many cases from a medical viewpoint. Therefore, if the length of a SOBP having a small dose distribution width is set to 1 cm, it is possible to form a SOBP having a desired length by superimposing SOBPs each having a small dose distribution width. In this way, the present invention provides an effect that a SOBP having a steep falling edge of the dose distribution on the deep side from the body surface and a desired length can be formed.

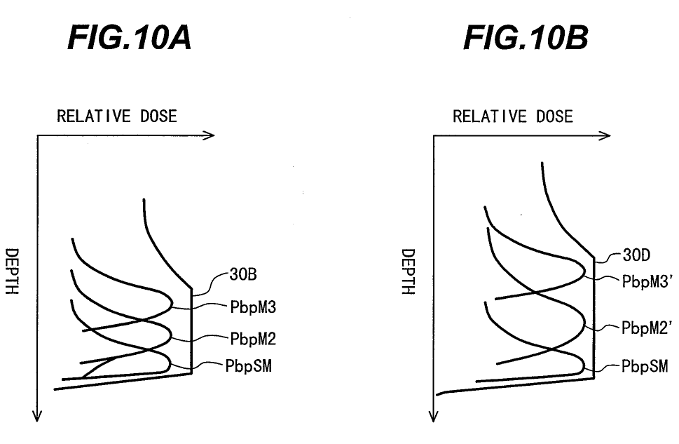

Then, the configuration and operations of a particle irradiation apparatus according to the second embodiment of the present invention will be explained below with reference to FIGS. 10A and 10B. The configuration of the particle irradiation apparatus according to the present embodiment is the same as that of FIG. 1. The present embodiment is characterized by the configuration of the energy spread device for producing a SOBP having a small dose distribution width.

FIGS. 10A and 10B are diagrams explaining SOBPs produced by the energy spread device for producing a SOBP having a small dose distribution width used for the particle irradiation apparatus according to the second embodiment of the present invention.

The spread width of a SOBP having a small dose distribution width to be produced can be changed by controlling the number, width, and height of layers of energy spread devices for producing a SOBP having a small dose distribution width. For example, the number and width of layers are increased for the energy spread device for producing a SOBP having a small dose distribution width in FIG. 3A to control their ratios to increase the width of the Bragg curve, thus producing a SOBP having a large spread width.

FIG. 10A is a diagram showing SOBPs formed using the energy spread device for producing a SOBP having a small dose distribution width in FIG. 4; FIG. 10B, a diagram showing a desired SOBP formed using SOBPs each having a large spread width.

For example, when the spread width of a SOBP having a small dose distribution width produced by the energy spread device M-RF for producing such a SOBP is 1 cm, the spread width of a SOBP having a small dose distribution width according to the present embodiment is set to 2 cm, 3 cm, or the like. When a combined SOBP having a 12-cm width is to be formed, for example, one SOBP (PbPSM) having a 1-cm width and a steep falling edge of the dose distribution on the deep side from the body surface is formed at the deepest portion of the target region from the body surface, and eleven SOBPs (PbpM2, PbpM3, and so on) each having a 1-cm width and a small dose distribution width are formed over the range from the second deepest portion to the shallowest target region on the body surface side. Thus, the formation of a SOBP having a small dose distribution width is repeated 12 times to form the combined SOBP having a 12-cm width.

In contrast, if SOBPs each having a large spread width are used as is the case with the present embodiment, one SOBP (PbpSM) having a 1-cm width is first formed at the deepest portion of the target region from the body surface. Then, for example, five SOBPs (PbpM2′, PbpM3′, and so on) each having a 2-cm width and one SOBP having a 1-cm width are formed over the range from the second deepest portion to the target region on the body surface side. Thus, the number of repetitions of SOBPs each having a small dose distribution width is reduced to seven when a SOBP 30D is to be formed.

Further, if one SOBP (PbpSM) having a 1-cm width is produced at the deepest portion of the target region from the body surface, and three SOBPs each having a 3-cm width and one SOBP having a 2-cm width are formed over the range from the second deepest portion to the shallowest target region on the body surface side, the number of repetitions of SOBPs each having a small dose distribution width is reduced to five when a combined SOBP is to be formed. In this way, in order to reduce the number of repetitions of SOBPs each having a small dose distribution width for forming a combined SOBP, it is possible to prepare one or a plurality of energy spread devices M-RF and one or a plurality of energy spread devices SM-RF, the SOBP widths produced by both of which differ, so as to reduce the number of repetitions.

In this way, in accordance with the present embodiment, the use of SOBPs each having a large spread width has an advantage that the number of repetitions of SOBPs each having a small dose distribution width can be reduced when a combined SOBP is to be formed. Specifically, this is effective in reducing treatment time. Further, because of the large widths of SOBPs, there is an effect that the flatness of a combined SOBP is not so largely affected even if the position of a SOBP having a small dose distribution width is shifted.

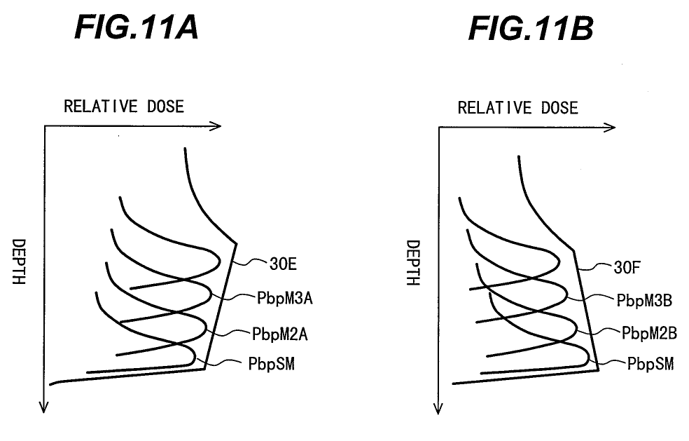

Then, the configuration and operations of a particle irradiation apparatus according to the third embodiment of the present invention will be explained below with reference to FIGS. 11A and 11B. The configuration of the particle irradiation apparatus according to the present embodiment is the same as that of FIG. 1. The present embodiment is characterized by the configuration of the energy spread device for producing a SOBP having a small dose distribution width.

FIGS. 11A and 11B are diagrams explaining SOBPs produced by the energy spread device for producing a SOBP having a small dose distribution width used for the particle irradiation apparatus according to the third embodiment of the present invention.

In a first example of the present embodiment, the shape of the dose distribution is changed. Here, the energy spread device for producing a SOBP having a small dose distribution width shown in FIG. 4 is employed; however, the proton beam intensity is increased, or the irradiation dose is controlled to produce SOBPs (PbpM2A, PbpM3A, and so on) each having a small dose distribution width and a large dose, as shown in FIG. 11A. The intensities of the SOBPs each having a small dose distribution width is increased to form a SOBP 30E having a larger dose on a shallower side from the body surface.

Further, in a second example of the present embodiment, the energy spread device for producing a SOBP having a small dose distribution width shown in FIG. 4 is employed; however, the proton beam intensity is decreased, or the irradiation dose is controlled to produce SOBPs (PbpM2B, PbpM3B, and so on) each having a small dose distribution width and a small dose, as shown in FIG. 11B. The intensities of the SOBPs each having a small dose distribution width is decreased to form a SOBP 30F having a smaller dose on a shallower side from the body surface.

As is the case with the present embodiment, it is possible to control the SOBP dose distribution by controlling the proton beam intensity or the irradiation dose to control the magnitude of the irradiation dose of a SOBP having a small dose distribution width. If the method of controlling the magnitude of the irradiation dose of a SOBP having a small dose distribution width by controlling the proton beam intensity or dose is applied to a SOBP having a large spread width, it is possible to control the SOBP dose distribution while reducing the number of repetitions of SOBPs each having a small dose distribution width.

In this way, an effect that the SOBP dose distribution can be controlled to a desired shape is obtained by controlling the magnitude of the irradiation dose of a SOBP having a small dose distribution width. This is effective, particularly in the case of a carbon beam, to form a SOBP having a steep falling edge of the dose distribution on the deep side from the body surface while giving an inclination to the SOBP dose distribution.

Next, the configuration and operations of a particle irradiation apparatus by a fourth embodiment of the present invention will be explained below. The configuration of the particle irradiation apparatus according to the present embodiment is the same as that of FIG. 1.

With the present embodiment, SOBPs each having a small dose distribution width are produced using the energy spread device for producing a SOBP having a small dose distribution width shown in FIG. 4; however, on the deep side of the target region from the body surface, the irradiation field is directly irradiated without allowing the beam to pass through an energy spread device. Specifically, the irradiation field is directly irradiated by allowing the proton beam to pass through the circular opening OP1 of the energy-spread-device section holder 42 shown in FIGS. 6 and 7.

Since the irradiation field is directly irradiated, the Bragg peak on the deep side of the target region from the body surface has a narrower spread, resulting in a steep falling edge of the dose distribution on the deep side from the body surface. From the second deepest portion of the target region, SOBPs each having a small dose distribution width are formed using the energy spread device for producing such a SOBP, thus forming a combined SOBP having a length suitable for the target region.

The present embodiment provides an effect that a SOBP can be formed through the combination of one type of energy spread device shown in FIG. 4 and direct proton-beam irradiation.

Further, this direct irradiation can also be used to irradiate a portion having an insufficient dose in order to ensure the flatness of the SOBP.

Although the irradiation apparatus and method of a proton-beam treatment system using a proton beam have been explained above, this irradiation method is applicable also to an irradiation apparatus of a particle treatment system using a beam of a heavy particle, such as carbon, helium, etc.

1. A particle irradiation apparatus for irradiating a target region with a particle beam, wherein:
the particle irradiation apparatus comprises a plurality of energy spread devices for forming high dose regions having different dose distribution shapes, and depth-directional dose distributions by said plurality of energy spread devices are combined.

2. A particle irradiation apparatus for irradiating a target region with a particle beam, wherein:
the particle irradiation apparatus comprises a plurality of energy spread devices having different geometric shapes, and depth-directional dose distributions by said plurality of energy spread devices are combined.

3. The particle irradiation apparatus according to claim 1, wherein:
said plurality of energy spread devices include:
a first energy spread device; and
a second energy spread device which forms a steeper dose distribution in the traveling direction of the particle beam than that formed by the first energy spread device.

4. The particle irradiation apparatus according to claim 3, further comprising:
a control unit which performs control so as to arrange the second energy spread device on a path of the particle beam when a layer at the deepest portion, out of a plurality of sectioned layers of the target region, is irradiated with the particle beam.

5. The particle irradiation apparatus according to claim 1, further comprising:
a monitor which measures an irradiation dose; and
a control unit which controls the irradiation dose;
wherein the particle irradiation apparatus performs:
sectioning the target region into layers;
determining at least an energy spread device and an irradiation dose used for each layer;
measuring an irradiation dose for each layer by means of the monitor; and
producing dose distributions with the irradiation dose controlled by the control unit and combining the produced dose distributions.

6. The particle irradiation apparatus according to claim 5, further comprising:
a first energy spread device which forms a first high dose region; and
a second energy spread device which forms a second high dose region at the deepest portion of the target region from the body surface;
wherein the particle irradiation apparatus performs:
forming the second high dose region at the deepest portion of the target region from the body surface through the use of the second energy spread device; and
forming the first high dose regions through the use of the first energy spread device once or a plurality of times over the range from the second deepest portion to the shallowest target region on the body surface side;
whereby the formed first and second high dose regions are superimposed to form a high dose region having a length suitable for the target region.

7. The particle irradiation apparatus according to claim 5, further comprising:
a range shifter which changes the irradiation depth of the particle beam;
a range-shifter drive unit which drives the range shifter; and
one or a plurality of energy types as particle beam energy of an accelerator;
wherein the particle irradiation apparatus performs:
forming high dose regions by changing the irradiation depth of the particle beam by use of either the range shifter driven by the range-shifter drive unit or the energy type or both and through the use of the first and second energy spread devices whereby the formed high dose regions are superimposed.

8. A particle beam irradiation method using the particle irradiation apparatus according to claim 6, the method comprising the steps of:
forming the second high dose region at the deepest portion of the target region from the body surface through the use of the second energy spread device; and
forming the first high dose regions through the use of the first energy spread device once or a plurality of times, over the range from the second deepest portion to the shallowest target region on the body surface side whereby the formed first and second high dose regions are superimposed to form a high dose region having a length suitable for the target region.

9. A particle beam irradiation method of irradiating a target region with a particle beam, the method comprising the steps of:
directly irradiating the target region with the particle beam to form a high dose region at a deep portion of the target region from the body surface;
irradiating with the particle beam the range from a deeper portion than said deep portion to the shallowest target region on the body surface side through the use of an energy spread device once or a plurality of times so as to form high dose regions whereby the formed high dose regions are superimposed to form a desired high dose region having a length suitable for the target region.

10. A particle treatment system, comprising:
an accelerator which accelerates a particle beam; and
a particle irradiation apparatus which receives the particle beam from the accelerator and irradiates a target region with the particle beam;
wherein the particle irradiation apparatus comprises:
a first energy spread device; and
a second energy spread device which forms a steeper dose distribution in the traveling direction of the particle beam than that formed by the first energy spread device;
wherein at least the first and second energy spread devices are employed to combine dose distributions in the traveling direction of the particle beam.

11. The particle treatment system according to claim 10, further comprising:
a control unit which performs control so as to arrange the second energy spread device on a path of the particle beam when a layer at the deepest portion, out of a plurality of sectioned layers of the target region, is irradiated with the particle beam.

12. The particle treatment system according to claim 10, wherein the particle beam is a proton beam.

13. The particle treatment system according to claim 10, wherein the particle beam is a heavy particle beam.
